(19) United States
(12) Patent Application Publication
(10) Pub. No.: US 2005/0208648 A1
(43) Pub. Date: Sep. 22, 2005

(54) Microdialysis needle assembly

(75) Inventors: Burton H. Sage JR., Hot Springs, AR (US); David Gillett, Rancho Bernardo, CA (US)

Correspondence Address: Burton Sage, Jr.;c/o Therafuse, Inc., 2453 Impala Drive, Carlsbad, CA, 92008, US

(73) Assignee: Therafuse, Inc.

(21) Appl. No.: 11/083,351

(22) Filed: Mar. 16, 2005

Related U.S. Application Data
(60) Provisional application No. 60/553,564, filed on Mar. 17, 2004.

(51) Int. Cl. C12M 1/34; A61K 9/22

(52) U.S. Cl. 435/287.2; 604/890.1

(57) ABSTRACT

A device for the measurement of analytes in a body fluid including a support with an upper surface, a layer adhered to the upper surface, the layer being of a hardenable material wherein at least one microfluidic channel has been etched after the layer is at least partially hardened, and a semipermeable membrane at least partially covering the layer.

This application claims priority to and subject matter disclosed in provisional application No. 60/553,564, filed on Mar. 17, 2004; the content of this application being incorporated by reference herein in its entirety. This application also claims subject matter disclosed in issued U.S. Pat. No. 6,582,393, issued Jun. 24, 2003, the contents of which are also incorporated by reference herein in their entirety.

### FIELD OF THE INVENTION

This invention relates to minimally invasive devices and methods for determining the concentration of compounds in body fluids of animals by means of microdialysis. Specifically, it relates to measurement of therapeutically useful analytes such as lactate and glucose in interstitial fluid.

### BACKGROUND

Microdialysis as a method of determining the concentration of compounds such as lactate and glucose in body fluids such as blood and interstitial fluid is well known. In 1987 Lonnroth, et al published “A microdialysis method allowing characterization of intercellular water space in humans” in the American Journal of Physiology 253:E228-E231. Further, in 1995, Stemberg, et al published “Subcutaneous glucose in humans: real time estimation and continuous monitoring” in Diabetes Care 18:1266-1269. The purpose of the efforts of Stemberg, et al, and the efforts and devices of many others working in the field of microdialysis, was to improve the measurement of glucose in blood and other body fluids, and thereby improve the quality of therapy for diabetes. In spite of these efforts, while significant progress has been made, there is yet no basis for a suitable product based on microdialysis.

Many products are currently marketed to measure blood glucose. One class of these products, known as glucose strips and meters, require a blood sample, usually from a fingertip. Strips and meters provide a satisfactory result when they are used, but they only provide a single result for each use. In diabetes, the glucose concentration in the body can change so quickly and so much that a single measurement, while being meaningful at the time it is taken, has little value even a short time later. In general, the more frequently the glucose concentration is measured, the better diabetes can be managed. From a practical point of view, though, a new and accurate glucose measurement with minimal time lag (delay caused by the time it takes to remove the specimen and make the measurement) every three to five minutes is adequate to effectively manage even the most brittle cases of diabetes.

This need for more frequent glucose measurements led to a second class of glucose measuring systems (known as “needle” sensors) that monitor glucose continuously. For over two decades, devices of this class, that measure glucose in a blood vessel or in interstitial fluid just below the surface of the skin, have been under development. Recently, such a device for use in interstitial fluid, developed by the MiniMed Corporation, was approved for sale. It can be used for up to three days.

This product, and other “needle sensors” currently under development, must be calibrated by a blood glucose measurement, usually obtained from fingerstick blood using a “strip and meter” device. The need for calibration is caused by a decrease in the sensitivity of the sensor to glucose over time during use. The sensor must be calibrated once when the product is first placed in the skin and, in the case of the approved product, as frequently as every eight hours until it is removed. While this system does provide superior glucose information, it is inconvenient for the user, who must both insert the needle and provide calibration as needed from fingerstick glucose measurements.

To avoid the decrease in sensitivity with time exhibited by the “needle sensors”, microdialysis systems for glucose were developed. These systems moved the actual glucose assay from the tip of the needle sensor, which is inside the body, to a place outside the body. This change of location resulted in a much more stable glucose sensitivity. However, a microdialysis system is more complicated than a needle sensor, and frequently requires perfusion of large volumes of fluid through the microdialysis needle, making the device too big for routine personal use. The volumes of fluids required for a day of use, for example, in the microdialysis system described by Pfeiffer in U.S. Pat. No. 5,640,954, were measured in hundreds of milliliters to liters per day.

Korf, in U.S. Pat. No. 6,013,029 describes an improved microdialysis system that uses much less fluid. In the preferred flow rate range specified by Korf, less than 20 microliters per hour, the amount of fluid required for a days use is less than 480 microliters, a volume that can be very comfortably worn. However, while Korf describes in detail the operation of his system, he does not provide details of construction of an interface which would be preferred for use in a microdialysis system.

Knoll, in U.S. Pat. No. 6,287,438 describes in broad detail many devices for use in sampling fluids for subsequent analysis, including devices that may be used in microdialysis systems. The systems of Knoll are comprised of laminates of multiple layers of materials each layer included to perform a specific function. The appeal of these devices is that each layer may comprise replicates of the pattern required for that layer, and that during manufacture, the several layers may be laminated in bulk, and the individual devices thus assembled cut out as finished devices.

Effenhauser, in U.S. Pat. No. 6,572,566 also describes a device comprised of several layers. Effenhauser, however, takes a broader approach than Knoll in that he includes in his device reservoirs for the reagents. Effenhauser, Korf, and Knoll all understand the need for smaller and more compact systems and each provides devices which consume minimal amounts of fluids thereby permitting an overall device size which may be conveniently worn by a user. Knoll, on the one hand, in U.S. Pat. No. 6,287,438, goes to great lengths to specify the materials that may be used in the construction of his device. Korf in U.S. Pat. No. 6,287,438 and Effenhauser in U.S. Pat. No. 6,572,566 on the other hand, while they discuss in great detail the many modes of operation of their devices, are quite vague on materials preferred for the construction of their devices.

Despite the advances described in U.S. Pat. No. 6,013,029, U.S. Pat. Nos. 6,287,438, and 6,572,566, there remain a number of unresolved issues that must be solved before a truly commercializable system is possible. The first such issue relates to body access. The sampling system, usually referred to as a microdialysis needle, must be constructed in such a way to permit easy and relatively painless placement of the needle in contact with the preferred body fluid. For easy and relatively painless body access, the support material must be extremely stiff so that it doesn't bend during insertion into body tissue, it must be able to bend without breaking, and it must be machinable so that a sharp point may be created for tissue penetration. Neither Korf nor Effenhauser describe materials preferred for body access, and none of the materials named by Knoll, that is the plastics, glass, ceramic nor silicon, are described as preferred for this purpose. Indeed—glass, ceramic and silicon are brittle, and subject to breaking upon skin penetration. And none of the plastics mentioned are capable of taking a sharp point for skin penetration.

A second unresolved issue relates to the size of the sampling system. While Korf dwells on overall system size, the dimensions of preferred interfaces are not detailed. And while Effenhauser does describe preferred channel dimensions and sampling fluid flow rates, preferred dimensions of the body access member are not described. Knoll does mention preferred dimensions of the body access member as being from 1 to 10 centimeters in length, 5 to 50 millimeters in width, and 0.1 to 1 millimeter in thickness. Even at the minimums of these dimensions, body access is almost certainly going to be painful.

A third unresolved issue relates to registration and alignment of the various layers during manufacture in the devices of Knoll and Effenhauser. Improper manufacture will result in the devices not functioning as described. Methods of assembly are not provided by Knoll, other than an occasional reference to adhesive bonding, although the methods of creating the various layers are listed exhaustively. When layers such as described are assembled, it is crucial that the various elements of the layers be aligned. Otherwise, for example, through holes may not line up with channels, and flow through the system may not occur or improperly occur.

Thus, there remains a need for improvements to provide a truly commercializable system.

### SUMMARY OF THE INVENTION

It is an object of at least some of the embodiments of this invention to provide a microdialysis system that may be easily and comfortably worn. The size of the system is such that the user may apply the system to the body using a skin adhesive and use the system for up to three days.

It is a second object of at least some of the embodiments of the invention to provide a microdialysis system which permits the user easy and comfortable body access. The dimensions and materials of the body access components are such that the user may perform the function with ease and a minimum of pain.

It is a further object of at least some of the embodiments of this invention is to provide a system design which permits cost-effective manufacturing and such that precise registration and alignment of the various components and layers is not required.

A microdialysis needle will be described wherein the needle substrate is made of a structural metal, and in one embodiment is made of stainless steel. Stainless steel is rigid enough, even in a small cross-section to permit skin penetration, it almost never breaks during insertion, may be machined to a sharp enough point that skin penetration is nearly painless, and is biocompatible.

The microdialysis needle of an exemplary embodiment of the current invention may be fabricated using photolithographic techniques. Using a structural metal support, a photoresist layer is added to the upper surface of the support. Using an appropriate pattern to create the necessary microfluidic pathways in the photoresist layer, this photoresist layer is exposed using standard photolithographic techniques. The photoresist layer is then partially hardened and etched using methods well known in the art. In one embodiment, the etching is such that channels in the photoresist layer are made completely through the photoresist layer. In one embodiment, the photoresist material is the epoxy SU-8 manufactured by MicroChem Corporation, Newton, Mass. After etching, a semipermeable membrane is placed on the partially hardened photoresist layer. The assembly is now heated further such that the partially hardened photoresist layer hardens further, adhering the semipermeable membrane to the photoresist layer. In a final step of one embodiment of the invention, the assembly is coated with a thin layer of an insulating material which will conformally coat the entire assembly. This coating material may be parylene which when applied by continuous vapor deposition, will conformally coat the assembly, including the microfluidic pathway beneath the semipermeable membrane, thereby creating a non-electrically conducting microfluidic channel.

### BRIEF DESCRIPTION OF THE FIGURES

FIG. 1 is an exploded view of a microdialysis sensor made according to an embodiment of the invention.

FIG. 2 is a drawing of the photolithographic mask with a magnified view of a section of the mask showing the layout of multiple microdialysis sensors on a substrate.

FIG. 3 is a scanning electron micrograph of a cross-section midway along the shaft of the microdialysis sensor made according to an embodiment of the invention.

FIG. 4 is a scanning electron micrograph of a cross-section at the tip of the microdialysis sensor made according to an embodiment of the invention.

FIG. 5 is a scanning electron micrograph of a test pattern showing the components of construction according to an embodiment of the invention.

### DETAILED DESCRIPTION

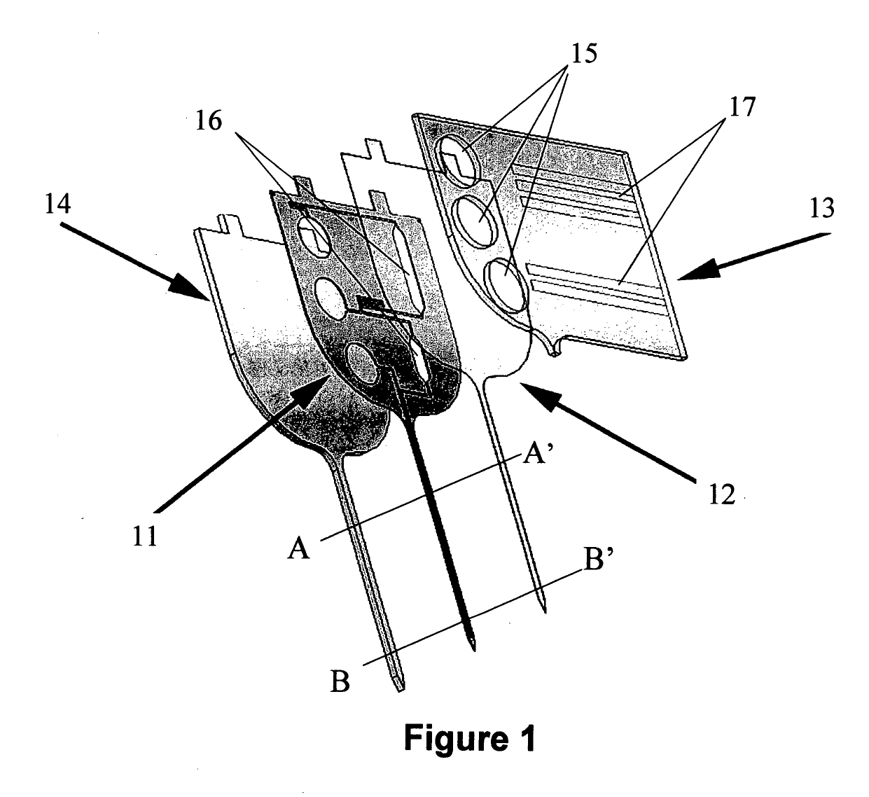

An exploded view of one embodiment of the invention is shown in FIG. 1. Support 14 is a structural material and is stainless steel in one embodiment. Layer 11 is adhered to structural layer 14 and comprises the microfluidic pathways and chambers required for the operation of the invention as a microdialysis sensor. Semipermeable membrane 12 is adhered to layer 11 covering the microfluidic pathways creating an integrated assembly of support, layer and membrane. This integrated assembly is then conformally coated with an insulating material (not shown). Cover 13 is then added to the assembly providing fluid access to the microfluidic network through access holes 15 and providing electrical access to chambers 16 through electrical leads 17.

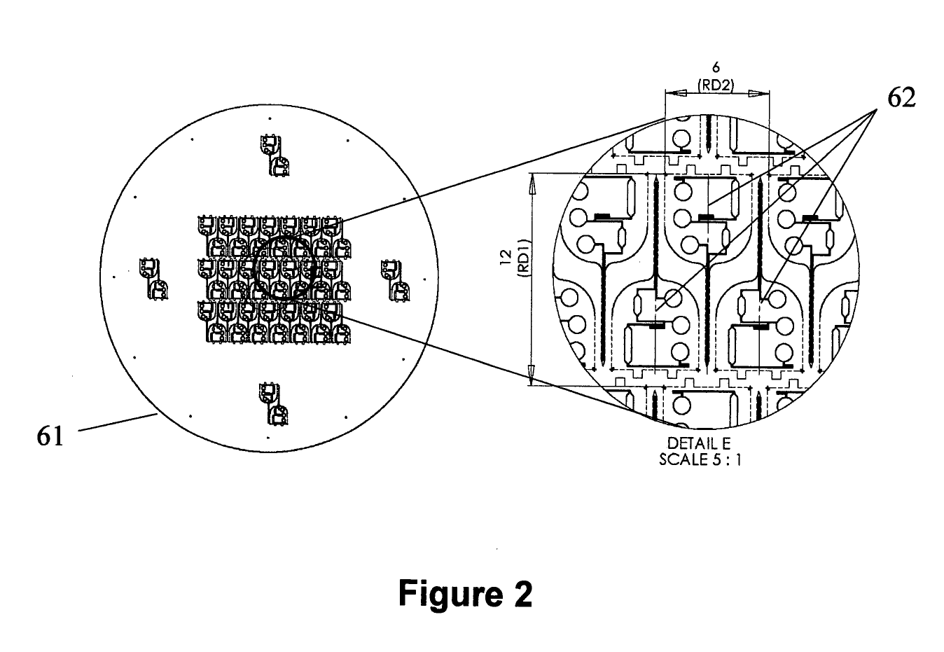

The device shown is FIG. 1 may be one of many identical devices on a single support as is shown, for example, in FIG. 2. Support 61 in FIG. 2 shows the many devices, with a magnified view showing the individual devices 62 on the support. While the number of devices that may be created on a single wafer will vary depending on the specific design of the device, at least 50 devices, and as many as 1000 devices will be fabricated on a single four inch wafer.

Support 14 of FIGS. 1 and 61 in FIG. 2 may be one in the same and may be one of several metals, but in one embodiment is stainless steel. Stainless steel has the required strength and biocompatibility for use in penetrating skin and for residing in tissue for several days. Layer 11 shown in FIG. 1 is added to support 14/61 by a method well known in the semiconductor industry. Layer 11 is a photoresist layer. The first step in adding layer 11 to support 14/61 is to clean the upper surface of support 14/61. In one embodiment, the cleaning step comprises placing support 14/61 in an oxygen plasma. This photoresist layer 11 is created on support 14/61 by placing support 14/61 in a photoresist spinner. The photoresist material in liquid form is added to the cleaned upper surface of support 14/61. In one embodiment, the photoresist is SU-8, but may be of any photoresist material which may be partially hardened in a first hardening step and subsequently exposed with a desired pattern for etching and then etched before a final hardening step.

To create layer 11 on support 14/61 in FIG. 2, the support 14/61 is spun at a high speed selected to create a uniform layer of liquid photoresist of a desired thickness. For the application of this invention, the thickness may range from about 5 microns to 500 microns.

The photoresist layer, still in liquid form but of uniform thickness, is then partially hardened. Partial hardening may be done by heating or other methods appropriate for the material.

To create the microfluidic network required for the sensor in layer 11, an example of which is shown in FIG. 1, a photolithographic mask of the microfluidic network is created. As shown in FIG. 2, this mask has many replicates of the desired microfluidic network. This mask is then imaged onto layer 11 and exposed using optical methods well known in the industry such that when the exposed photoresist layer is developed, the microfluidic network will be etched into layer 11. When layer 11 is SU-8, the developer may be selected from polyglycol methyl ether acetate (PGMEA) or equivalents. However, when the photoresist material for layer 11 is selected, preferred etchants can also be selected from those appropriate for the selected photoresist material as is well known in the semi-conductor industry. The channels and chambers of the microfluidic network will all be etched to the same depth by this process. The selected depth may be a portion of the thickness of layer 11 but may also be the entirely of the thickness of layer 11. Since the etchant will not etch the metal support, the etching step may be of sufficient time to etch completely through layer 11 to avoid variations in etching depth due at different locations support 14/61 which may occur due to variations in etching efficiency due to temperature and potency differences in the etching bath.

To complete the basic assembly of the microdialysis sensor, a semi-permeable membrane 12 is placed over the microfluidic network in photoresist layer 11. Alignment of membrane 12 with respect to the microfluidic network is not critical in some embodiments. In the needle portion of the sensor, the microfluidic network comprises a microdialysis channel that combines with membrane 12 to form an analyte exchange region. This is the region of the sensor that will be in contact with body fluid. In this analyte exchange region, the body analyte is able to move across membrane 12 in either direction, that is, into or out of the microdialysis channel. In the analysis section of the sensor, the membrane may be removed as described later, or may be left intact to protect electrodes.

The membrane 12 is adhered to the partially hardened layer 11 by simply placing the membrane in contact with layer 11 and heating the partially assembled sensor further. In one embodiment where the photoresist layer is SU-8 and the membrane is a polycarbonate film with pores created by the track etch method, this heating further hardens layer 11 and causes strong adherence of the membrane to the photoresist layer. The track-etch method of formation of a semipermeable membrane is well known in the field. The Whatman-Nuclepore website provides a description of this method. The content of this site is incorporated herein by reference. An advantage of track-etch membranes in this use is that these membranes do not permit lateral motion of fluids—the only motion is through the membrane-thereby preventing leakage from one part of the microfluidic network to another.

To seal the assembly, the sensor at this stage of assembly is conformally coated with an insulator (not shown). In one embodiment of the assembly process, this insulator is provided by continuous vapor deposition. In this process, the insulating material is provided in a vapor phase in a chamber that also contains the objects to be coated. When the insulator material is parylene, the coating can be conformal over the entirely of the object, including the metallic bottoms of the channels and chambers of microfluidic network which are exposed if the etching process of layer 11 is a through etch. An advantage of selecting parylene as the insulator is that conformal coatings of parylene formed by the continuous vapor deposition process can be pinhole free at thicknesses of 10 nanometers or more. At this insulator layer thickness, the pores in the track-etch membrane, which may have a diameter as small as 50 nanometers, are not filled during the insulator deposition process but are merely slightly reduced in diameter.

The next step in the fabrication method is to cut out the microfluidic elements from the support. This may be easily done by laser cutting as is known in the industry, but may also be done by die cutting or other such methods. The process of cutting out the microfluidic elements as shown in FIG. 2 may not require high positional tolerances for some embodiments of the invention, as would be needed to align the layers of the sensor if the individual layers were created separately and the sensor created by a layering method. During this cutting out step, the membrane may be removed from the portions of the microfluidic network that provide fluid access or electrical access.

The final step in the creation of the microdialysis sensor according to the invention is to add manifold layer 13 to the insulator coated membrane surface of the assembled microfluidic network. Fluid access ports 15 are provided to supply the required fluids to operate the sensor and electrical access leads 17 are provided for the electrochemical aspects of the sensor. Fluid access ports 15 are shown so that they align with similar chambers in photoresist layer 11 in FIG. 1. Not shown are the fluidic interconnects to fluid supply reservoirs which are not part of the microdialysis sensor according to some embodiments of the invention.

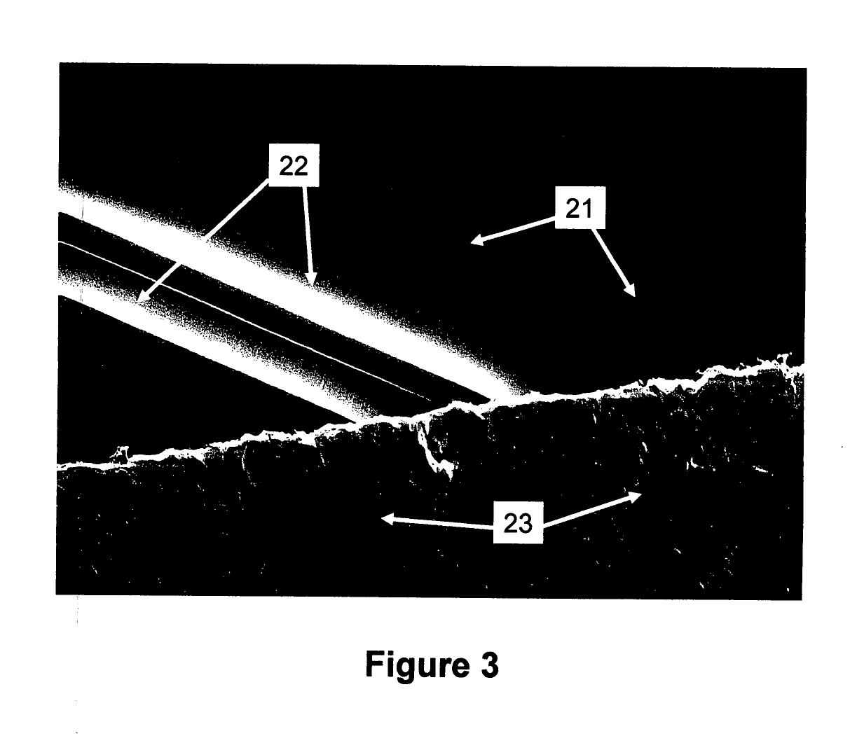

FIG. 3 is a scanning electron photomicrograph of the section of the exchange region of the microdialysis sensor shown in FIG. 1 designated by section A-A′. A portion of semipermeable membrane 23 has been removed to expose channel 22 which has been etched in photoresist layer 21. As can be seen, Channel 22 has been etched completely through photoresist layer 21 so that the bottom of channel 22 is the metal support layer.

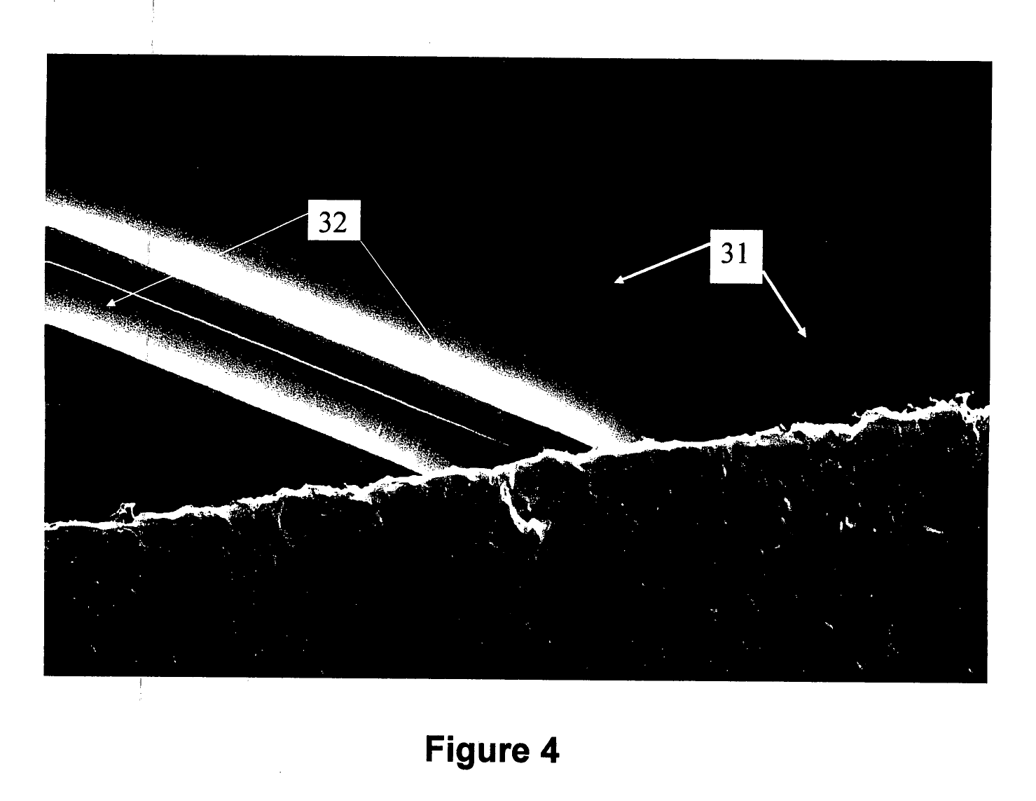

FIG. 4 is a scanning electron photomicrograph of the section of the exchange region of the microdialysis sensor shown in FIG. 1 designated by section B-B′. Again, a portion of the semipermeable membrane has been removed to expose channel 32 which has been etched in photoresist layer 31, and the channel 32 has been etched completely through photoresist layer 31 to expose the metal support.

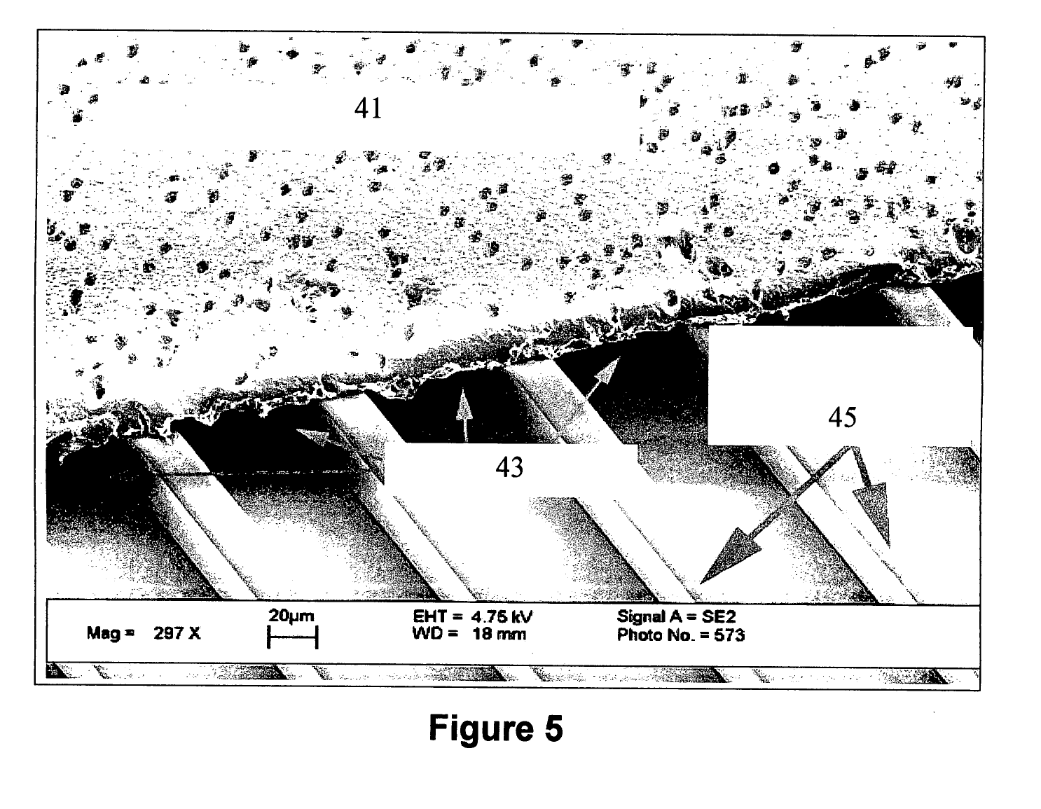

FIG. 5 is a scanning electron photomicrograph of a test microfluidic network of the various materials and processes used to make the microdialysis sensor. In FIG. 5, the pores in the semipermeable membrane 41 can be clearly seen. Also, the channels 43 formed in the photoresist layer 45 are more clearly shown. As above, the photoresist layer has been etched completely through and the metal support of the bottom of channels 43 can clearly be seen.

As may be seen by FIGS. 3, 4, and 5, the quality of the channels formed by this technique is high. In these figures, dimensions that would be typical but not limiting for a microdialysis sensor are shown. The channels are 50 microns wide by 20 microns deep. The semipermeable membrane is 10 microns thick. The support layer is 150 microns thick. These dimensions provide overall dimensions of the body penetrating portion of the microdialysis sensor of less than 200 microns by 200 microns, or similar to a 32G cannula.

The microdialysis sensor of some embodiments of the present invention operates in the following exemplary manner (for a more complete description of the operation of this sensor and operational variants, see copending patent application Ser. No. 10/059,390 entitled Self-Calibrating Body Analyte Monitoring System filed Jan. 31, 2002 which is incorporated herein in its entirely by reference. Perfusate is introduced into the sensor through the lower of fluid access ports 15 in FIG. 1. After entering the sensor, the perfusate flow through the exchange region of the sensor and emerges into the lower of reaction chambers 16 as dialysate. In reaction chamber 16, the dialysate is exposed to an electric field which removes electrochemically active compounds which may interfere with analysis of the desired body analyte. The electric field may be of sufficient strength to electrolyze water thereby adding oxygen to the fluid for use in a subsequent enzymatic reaction if needed. After flowing through lower chamber 16, the oxidized dialysate is mixed with an enzyme solution that is provided through the center access port 15. As an example, the desired body analyte may be glucose, which is not electrochemically active, and the enzyme solution may be glucose oxidase. The mixing of the glucose and the glucose oxidase creates glucuronic acid and hydrogen peroxide as is well known in the industry. With its glucose now converted to hydrogen peroxide and glucuronic acid, the dialysate now proceeds to the upper reaction chamber 16 where hydrogen peroxide is electrochemically reduced to water and oxygen, providing a measurement of the original glucose content of the body tissue where the exchange region of the sensor is located. After this electrochemical measurement, the dialysate proceeds to the upper fluidic access port 15 and out of the sensor to waste.

The body analyte sensor according to some embodiments of the present invention may be used to monitor or measure the concentration of many compounds in many solutions and is not limited to the above example of monitoring the concentration of glucose in a body fluid such as interstitial fluid. Other endogenous compounds such as lactate, or pharmaceutical agents such as theophylline in the treatment of asthma or various anticoagulants in the treatment of thrombosis may be measured. And other body fluids such as blood or cerebral spinal fluid may be sampled using this sensor. Further, at least some of the embodiments of this invention may be used with a self-calibrating system.

In view of the above, some embodiments of the Body Analyte Monitoring System Assembly as described above and/or according to other embodiments of the present invention may be used in combination with one or more of the embodiments of the drug delivery systems described in U.S. application Ser. No. 10/146,588 dated May 15, 2002 and/or U.S. application Ser. No. 10/600,296 dated Jun. 20, 2003, and/or copending application Ser. No. 10/059,390, filed Jan. 31, 2002, and/or U.S. application Ser. No. 09/867,003 filed May 29, 2001, now U.S. Pat. No. 6,582,393, issued Jun. 24, 2003, and/or U.S. application Ser. No. 10/662,871 dated Sep. 16, 2003, and/or copending application number and/or copending application Ser. No. 10/786,562 filed on Feb. 26, 2004, and/or provisional application No. 60/553,564 filed on Mar. 17, 2004. Thus, some embodiments of the present invention include the combination of a body analyte monitoring system/self-calibrating body analyte monitoring system utilizing the Body Analyte Monitoring System Assembly as disclosed herein in combination with a drug delivery system, which may be, by way of example and not by way of limitation, in a single integrated system and/or in two or more quasi-separate systems in communication with each other which may, again by example, be worn or otherwise carried by a user. In such embodiments, a Body Analyte Monitoring System Assembly as described herein may be utilized in or with a body analyte monitoring system/self-calibrating body analyte monitoring system to monitor a body analyte and/or a drug delivery system to control the amount/rate/dosage, etc., of drug delivered to the user based on the results of monitoring by the body analyte monitoring system utilizing the Body Analyte Monitoring System Assembly. Thus, in some embodiments, a device/method may be manufactured/used where the two systems/assemblies work together to ensure/help ensure that a patient receives proper/adequate amounts of a beneficial drug.

While specific embodiments of the invention have been described in detail, it will be appreciated by those skilled in the art that various modifications and alternatives to those details could be developed in light of the teaching of the disclosure. Accordingly, the particular embodiment described in detail is meant to be illustrative and not limiting as to the scope of the invention, which is to be given the full breadth of the appended claims and any and all equivalents thereof.

1. A device for the measurement of analytes in a body fluid comprising:
a. a support with an upper surface,
b. a layer adhered to the upper surface, the layer including a hardenable material wherein at least one microfluidic channel has been etched after the layer is at least partially hardened, and
c. a semipermeable membrane at least partially covering the layer.

2. The device of claim 1 wherein the support is metal.

3. The device of claim 2 wherein the metal is stainless steel.

4. The device of claim 1 wherein the hardenable material is a photoresist.

5. The device of claim 4 wherein the photoresist is an epoxy.

6. The device of claim 5 wherein the epoxy is SU-8.

7. The device of claim 1 wherein the microfluidic channel is etched completely through the layer.

8. The device of claim 1 wherein the semipermeable membrane is a track-etched semipermeable membrane.

9. The device of claim 1 conformally coated with an insulating layer.

10. The device of claim 9 wherein the conformal coating is a vapor deposited coating.

11. The device of claim 10 wherein the conformal coating is parylene.

12. The device of claim 1, wherein the device includes a plurality of microfluidic channels etched after the layer is at least partially hardened, the plurality of microfluidic channels forming a microfluidic network, further comprising a manifold covering at least a portion of the semipermeable membrane.

13. The device of claim 12, wherein the manifold comprises:
a plurality of fluid access ports adapted to provide fluid access to the microfluidic network to permit a plurality of respective fluids to at least one of enter and exit the microfluidic network through the manifold.

14. The device of claim 13, wherein the manifold further comprises electrical leads adapted to provide electrical access to chambers of the microfluidic network.

15. A body analyte measurement system, comprising:
a. a first reservoir for containing a solution comprising a known concentration of the body analyte;
b. a second reservoir for containing an enzyme solution;
c. the device of claim 14, wherein the manifold includes an inlet in liquid communication with the first reservoir and an outlet, wherein the device is adapted to allow exchange of the body analyte between the solution and a body fluid when there is solution in the device and the device is in contact with the body fluid, wherein the device further includes a measurement path comprising a first chamber, a second chamber downstream from the first chamber, and a third chamber downstream from the second chamber such that the first chamber may receive liquid from the first reservoir and the outlet and the second chamber may receive liquid from the first chamber and the second reservoir; and
d. a valving system for controlling liquid flow along the measurement path such that the liquid flowing into the second chamber is either (i) liquid from the outlet that has passed through the first chamber and liquid from the second reservoir, or (ii) liquid from the first reservoir that has passed through the first chamber and liquid from the second reservoir.

16. The system of claim 15, wherein the valving system is contained inside the device.

17. A method of making a microdialysis sensor comprising:
a. providing a support with an upper surface,
b. creating a layer on the upper surface by adding a hardenable liquid to the upper surface and partially hardening the hardenable liquid such that the hardenable liquid adheres to the upper surface and forms a new upper surface,
c. creating a channel in the partially hardened layer,
d. adhering a semipermeable membrane to the new upper surface and hardening the layer to form an assembly including the support, the layer and the membrane, and
e. conformally coating the assembly with an insulating layer.

18. The method of claim 17 wherein the support is metal.

19. The method of claim 18 wherein the metal is stainless steel.

20. The method of claim 17 wherein the hardenable material is a photoresist.

21. The method of claim 20 wherein the photoresist is an epoxy.

22. The method of claim 21 wherein the epoxy is SU-8.

23. The method of claim 17 wherein the microfluidic channel has been etched completely through the layer.

24. The method of claim 17 wherein the semipermeable membrane is made by the track-etch method.

25. The method of claim 17 wherein the conformal coating is applied by vapor deposition.

26. The method of claim 25 wherein the conformal coating is parylene.

27. The method of claim 18, further comprising attaching a manifold to the assembly.

28. A method of making a microdialysis sensor comprising:
a. providing a metal support with an upper surface,
b. treating the upper surface using oxygen plasma,
c. creating a layer on the prepared upper surface by adding a hardenable liquid to the prepared upper surface and partially hardening the hardenable liquid such that the hardenable liquid adheres to the prepared upper surface and forms a new upper surface,
d. creating a channel in the partially hardened layer,
e. adhering a semipermeable membrane to the new upper surface by contacting the semipermeable membrane to the partially hardened layer and heating the assembly to further harden the layer to form an assembly including the support, the layer and the membrane, and
f. conformally coating the assembly with an insulating layer.

29. The method of claim 28 wherein the metal is stainless steel.

30. The method of claim 28 wherein the hardenable liquid is SU-8.

31. The method of claim 28 wherein the semipermeable membrane is made using the track-etch method.

32. The method of claim 28 wherein the insulating layer is parylene.

33. The method of claim 28 wherein the metal is stainless steel, the hardenable material is SU-8, the semipermeable membrane is made by the track-etch method, and the insulating layer is parylene.

34. The method of claim 28, further comprising attaching a manifold to the assembly.

35. A device comprising a plurality of body analyte monitoring system assemblies, the assemblies comprising:
a. a support with an upper surface, wherein the support with an upper surface is shared by the plurality of the assemblies;
b. a layer adhered to the upper surface, the layer including a hardenable material wherein a plurality of microfluidic channels are etched after the layer is at least partially hardened, the plurality of microfluidic channels forming a plurality of respective microfluidic networks of the assemblies, wherein the layer is shared by the plurality of assemblies;
c. a semipermeable membrane at least partially covering the layer, wherein the membrane is shared by the plurality of assemblies; and
d. manifolds associated with respective assemblies, the manifolds covering at least a portion of the semipermeable membranes of the respective assemblies, wherein the manifolds comprise a plurality fluid access ports adapted to provide fluid access to the microfluidic network of the respective assemblies to permit a plurality of respective fluids to at least one of enter and exit the respective microfluidic network through the respective manifold, and wherein the manifolds further comprises electrical leads adapted to provide electrical access to respective chambers of the respective microfluidic network.

36. The device of claim 35, wherein the device comprises at least about 100 assemblies.

37. The device of claim 35, wherein the device comprises at least about 500 assemblies.

38. A method of making a plurality of microdialysis assemblies, comprising the actions of:
a. providing a support with an upper surface;
b. creating a layer on the upper surface by adding a hardenable liquid to the upper surface and partially hardening the hardenable liquid such that the hardenable liquid adheres to the upper surface and forms a new upper surface;
c. creating a plurality of channels in the partially hardened layer to form a plurality of individual microfluidic networks;
d. adhering a semipermeable membrane to the new upper surface and hardening the layer to form an assembly including the support, the layer and the membrane;
e. creating a manifold layer, the manifold layer comprising a plurality fluid access ports adapted to provide fluid access to the individual microfluidic networks to permit a plurality of respective fluids to at least one of enter and exit the respective microfluidic networks through the manifold layer, and wherein the manifold layer further comprises electrical leads adapted to provide electrical access to chambers of the microfluidic networks; and
f. separating the device made by actions a-e into a plurality of assemblies, the plurality of assemblies including a plurality of assemblies each including a microfluidic network, and a plurality fluid access ports adapted to provide fluid access to the microfluidic network to permit a plurality of respective fluids to at least one of enter and exit the microfluidic network through the manifold.

39. The method of claim 38, further comprising separating the device into at least about 100 assemblies.

40. The device of claim 1 further comprising a drug delivery system such that the amount or rate of delivery of the drug by the drug delivery system is based on a measurement made by the device.

41. The device of claim 35 further comprising a drug delivery system such that the amount or rate of delivery of the drug by the drug delivery system is based on a measurement made by the device.
